(19) United States
(12) Patent Application Publication
(10) Pub. No.: US 2025/0185903 A1
(43) Pub. Date: Jun. 12, 2025

(54) ADAPTER FOR CONNECTING A PORT OF A CHANNEL OF A SURGICAL INSTRUMENT TO A CLEANING FLUID SUPPLY

(71) Applicant: OLYMPUS Winter & Ibe GmbH, Hamburg (DE)

(72) Inventor: Sascha ESCHBORN, Ahrensburg (DE)

(73) Assignee: OLYMPUS Winter & Ibe GmbH, Hamburg (DE)

(21) Appl. No.: 18/976,762

(22) Filed: Dec. 11, 2024

(30) Foreign Application Priority Data
Dec. 12, 2023 (DE) 10 2023 134 851.5

(51) Int. Cl. A61B 1/12; A61B 1/00

(52) U.S. Cl. CPC A61B 1/125; A61B 1/00119

(57) ABSTRACT

An adapter for connecting a port of a channel of a surgical instrument to a cleaning fluid supply. The adapter including a rigid housing with a port interface and a connection interface for the cleaning fluid supply. Wherein the port interface comprises a gasket with a central opening for guiding the fluid into the port of the channel, the port interface with the gasket is configured to be pressed along a pressing direction on the port of the channel to establish a sealing and rinse the channel with the cleaning fluid, and the gasket is deformable along the pressing direction in order to fit differently configured ports.

### CROSS-REFERENCE TO RELATED APPLICATION

The present application is based upon and claims the benefit to DE 10 2023 134 851.5 filed on Dec. 12, 2023, the entire contents of which are incorporated herein by reference.

### BACKGROUND

### Field

The present disclosure relates to an adapter for connecting a port of a channel of a surgical instrument to a cleaning fluid supply.

### Prior Art

After each use, surgical instruments, for example endoscopes, have to be cleaned and disinfected before they can be reused. This is called the reprocessing of surgical instruments.

As part of the reprocessing of surgical instruments like flexible endoscopes, the channels of the surgical instruments need to be rinsed with a cleaning fluid in a water bath or a similar device. The cleaning fluid may for example be normal water, a mixture of water and a dosed cleaning agent or a mixture of air and water. This cleaning fluid is rinsed through the channels of the surgical instrument with a specific pressure, flow rate and pulse rate. With the help of semi-automatic rinsing devices, a combination of all three cleaning fluids may be used during the reprocessing process.

However, surgical instruments often feature a number of different channels, each with different shaped ports and different sized port openings. In order to connect a rinsing device to these channels, different shaped adapters are required for each channel and each different type of surgical instrument. Often a user needs twenty or more different adapters to be able to flush all the different channels of their portfolio of surgical instruments. In addition, some surgical instruments comprise up to seven different ports. Thus, the adapters utilized to connect to these surgical instruments require up to seven port interfaces. The setting and reconnection of all these port interfaces is time consuming and prone to mistakes.

### SUMMARY

An object is to provide an adapter for connecting all ports of a surgical instrument to a cleaning fluid supply, which simplifies the process of cleaning the channels in a water bath.

Such object can be solved by an adapter for connecting a port of a channel of a surgical instrument to a cleaning fluid supply, the adapter comprising a rigid housing with a port interface and a connection interface for the cleaning fluid supply, wherein the port interface comprises a gasket with a central opening for guiding the fluid into the port of the channel, wherein the port interface with the gasket is configured to be pressed along a pressing direction on the port of the channel to establish a sealing and rinse the channel with the cleaning fluid, wherein the gasket is deformable along the pressing direction in order to fit different ports.

The adapter can be an universal adapter configured to fit different ports of all shapes and sizes on a surgical instrument. A diameter of the port interface can be larger than the largest port, which it is configured to fit. This can allow the adapter to fit on all ports of different surgical instruments. The gasket can establish a sealing with the port in order to efficiently rinse the channel. In order to fit all kinds of ports, the gasket is deformable along the pressing direction. This allows the gasket to enclose the port opening of the channel, regardless of the shape and size of the port.

The pressing direction can be parallel to a central axis of the housing. A flow direction of the cleaning fluid inside the port interface can be parallel to the pressing direction. The pressing direction is the direction, in which the adapter is pressed on the port.

The housing may be made of metal or plastic. The housing can be a rotary milled body. The housing may be hollow. The housing may have a first opening at a first base area and/or a second opening at a second base area opposite the first base area. The housing may be made of a one piece. The housing may enclose the gasket in a circumferential direction. The surgical instrument may be, for example, a flexible endoscope.

According to an embodiment, the gasket can comprise a flexible sealing lip, wherein the flexible sealing lip can be configured to curve itself around the port opening of the port. The form of a flexible sealing lip can be configured to fit different ports. In addition to be deformable along the pressing direction, the flexible sealing lip may also be deformable in a radial direction. The radial direction extends from the center of the central opening outwards and is orthogonal to the pressing direction. A thick gasket may be employed in order to increase the accuracy when pressing the gasket on the port. Thin gaskets may have a higher deformability along the pressing direction.

The gasket may be rotationally symmetrical. This simplifies the pressing of the gasket on the port, as the symmetry will help center the gasket on the port.

The flexible sealing lip can comprise an outer sealing surface and an inner sealing surface, the outer sealing surface surrounding the inner sealing surface and the inner sealing surface surrounding the central opening in a circumferential direction. The two different sealing surfaces, the outer sealing surface and the inner sealing surface, can increase the flexibility of the sealing lip, as they may be deformed in different directions. The circumferential direction can be orthogonal to the pressure direction and/or the radial direction.

The outer sealing surface can be curved in an opposite direction as the inner sealing surface. This can increase the flexibility of the sealing lip and can achieve a better fit to different ports. The flexible sealing lip may be a solid of revolution, wherein an axis of revolution is the central axis of the housing.

Each of the outer sealing surface and the inner sealing surface can be conical frustum shaped, such that a transition between the outer and inner sealing surfaces forms concavity. The combination of the conical frustum shapes can allow the sealing lip to adapt to a great number of different port sizes and shapes. The outer sealing surface can be conical frustum shaped and the inner sealing surface can be conical frustum shaped.

The outer sealing surface can be surrounded by a ring shaped trim, wherein the inner sealing surface can protrude in the pressing direction from the trim and/or the outer sealing surface can be recessed compared to the trim. The gasket may be fixed to the housing by the trim. The trim may form the outermost part of the sealing lip.

The central opening can be formed as a gasket channel extending in the pressing direction, wherein a wall of the gasket channel can be flexible in a radial direction of the gasket. The wall of the gasket channel can be an inner wall surrounding the central opening. By having a flexible wall, the gasket channel may wrap around the outer surface of small ports. For larger ports, the entire inner sealing surface, and even the outer sealing surface, may be arranged inside the port opening when the adapter is pressed on the port. The inner sealing surface may protrude from the trim and the outer sealing surface may be recessed compared to the trim, allowing the sealing lip to fit different port sizes even better. The gasket channel may widen towards the central opening.

According to an embodiment, the gasket can comprise an elastic seal with a toroidal shape. The elastic seal with a toroidal shape can allow the gasket to adapt its form to the port of the surgical instrument. The elastic seal can be made of a soft material. The soft material can be rubber with a shore hardness from 25 to 65. This can allow the elastic seal to form a sealing with ports of very different shapes and sizes. The elastic seal may be fixed to the housing with an endplate. The endplate may overlap a radially extending part of the elastic seal in order to hold the elastic seal.

A height of a cross section of the elastic seal can be larger than a width of the cross section. The height of the cross section can extend parallel to the central axis of the housing. The width of the cross section can extend parallel to the radial direction of the gasket. In other words, the elastic seal can be very thick. The elastic seal can also be very voluminous in order to be very compressible. The height of the cross section can be at least one and a half times as large as the width of the cross section. The cross section of the elastic seal may be non-symmetrical. The toroidal shape of the elastic seal can give the elastic seal a doughnut-like shape. However, unlike a doughnut, the cross section does not have to be circular, but extended in the pressing direction.

The elastic seal can be compressible in the pressing direction to less than 80%, such as less than 50%, of its length. This compressibility can allow the elastic seal to fit a great number of different ports. The length is the extension of the elastic seal along the pressing direction.

The adapter can comprise a rigid tube extending through the central opening of the gasket. The rigid tube can provide stability to the central opening. This can be especially important when utilizing an elastic seal with a toroidal shape, as it can prevent the central opening from becoming narrowed or completely blocked when the elastic seal is compressed and can provide the cleaning fluid with the necessary flow rate. The adapter may comprise a guiding sleeve arranged inside the housing and inside the elastic seal. The guiding sleeve surrounds the rigid tube at least partially and holds it in place.

The adapter can comprise a spring connecting the housing to the gasket, wherein the spring can be configured and arranged to absorb a force resulting from pressing the gasket on the port of the channel, wherein the spring can be a flat spring. The spring can prevent the adapter from being damaged when it is pressed on a port with excessive force. It can also help to find the right amount of pressing force. The spring may be arranged inside the housing above the gasket in the pressing direction.

The adapter may comprise a notification device, which can be configured to output an acoustic signal when the gasket is pressed on the port of the channel with a force and/or pressure exceeding a predetermined threshold. The notification device can signal a user through an acoustic signal that a sufficient sealing has been achieved and the adapter does not need to be pressed any harder on the port. For example, the notification device may be a loudspeaker. The force may be determined through the spring or another force measuring device or a pressure measuring device.

The gasket can be made of or comprises silicone and/or plastic and/or rubber. The gasket, such as the elastic seal, may be formed from a rubber composite part. The sealing lip may be formed of silicone. These materials can be advantageous as they can offer elasticity and compressibility and they can also be sufficiently waterproof.

The sealing established by the port interface and the port can only be partially tight to water, wherein a tightness of the sealing to water can be between 80% and 100%, such as between 80% and 90%. By being only partially tight to water, some of the cleaning fluid will exit from the sealing and flush the contact surfaces of the adapter and the port. This can allow an additional cleaning of these contact surfaces without requiring an additional work step. In other words, the sealing created by the flexible sealing lip and/or the elastic seal may be permeable to water. This configuration can improve the cleaning process.

The cleaning fluid may be supplied through the connection interface via a cleaning fluid connection. The cleaning fluid connection can connect the adapter to a cleaning fluid supply, for example a tank of cleaning fluid. The cleaning fluid may be water or a mixture of water and a cleaning agent and/or a mixture of water and air.

The housing can have a cylindrical shape, wherein the housing can have the shape of a first cylinder connected to a second cylinder, such as by a conical frustum, wherein a diameter of the first cylinder can be larger than a diameter of the second cylinder. A cylindrical shape can make the adapter easy to handle for a user and easy to fit different ports. A configuration with a first cylinder and a smaller second cylinder can give enough space for the adapter to be gripped and for the connection interface in the first cylinder, while simultaneously providing an adequately sized port interface in the second cylinder. Alternatively, the housing may be shaped as a single cylinder.

The port interface can be arranged in a first base area of the housing. The first base area is an end face of the housing arranged on a first end of the adapter. According to an embodiment, the port interface can be arranged in a base of the second cylinder opposite the conical frustum.

The connection interface for the cleaning fluid supply can be arranged on a lateral surface of the housing. This arrangement provides enough room for the connection interface. The connection interface can be configured to connect a tube, pipe or hose to the adapter. The adapter can be supplied through this tube, pipe or hose with the cleaning fluid. According to an embodiment, the connection interface for the cleaning fluid supply can be arranged on a lateral surface of the first cylinder or the second cylinder.

The housing can comprise a pressure receiving area at a second base area of the housing opposite the port interface, the pressure receiving area can be configured to receive a pressing force to press the gasket on the port of the channel. The pressure receiving area can be configured to provide a user with the surface to press the adapter along the pressing direction on the port. The pressure area may can also be configured to hold the adapter in front of the port and align it according to the flow or pressure angle. According to an embodiment, the pressure receiving area can be arranged on a base of the first cylinder opposite the conical frustum. The pressure receiving area may comprise with one or more holding devices. The holding devices may be configured to allow the user to grip the adapter.

According to an embodiment, the pressure receiving area can be configured as a robot arm interface. The robot arm interface can be configured to receive a robot arm, which may press the adapter on the port. The robot arm interface may comprise one or more of the holding devices, wherein the holding device of the robot arm interface may be configured to connect the adapter to the robot arm. This allows for a semi-automatic cleaning. The user can only arrange the adapter on the port, while the pressing can be done be the robot arm. Instead of a robot arm, there may also be a different automatic pressing device.

The adapter can comprise an inner channel extending inside the housing, the inner channel connecting the connection interface for the cleaning fluid supply with the port interface, wherein a diameter of the inner channel can decrease towards the port interface. The inner channel can guide the cleaning fluid from the connection interface to the port interface. By decreasing the diameter of the inner channel towards the port interface, the inner channel can act as a nozzle, increasing the flow rate of the cleaning fluid.

According to an embodiment, the diameter of the inner channel at a position at the port interface can be less than half, such as less a third as big as the diameter of the port interface.

According to an embodiment, the adapter can comprise a fixing device, wherein the fixing device can be configured to detachably fix the port interface to the port of the channel. The fixing device may be formed to pin, plug, screw or otherwise attach the adapter to the port.

Further features will become evident from the description of embodiments, together with the claims and the appended drawings. Embodiments can fulfill individual features or a combination of several features.

The embodiments described below, without restricting the general intent of the invention, based on exemplary embodiments, wherein reference is made expressly to the drawings with regard to the disclosure of all details that are not explained in greater detail in the text.

In the drawings:

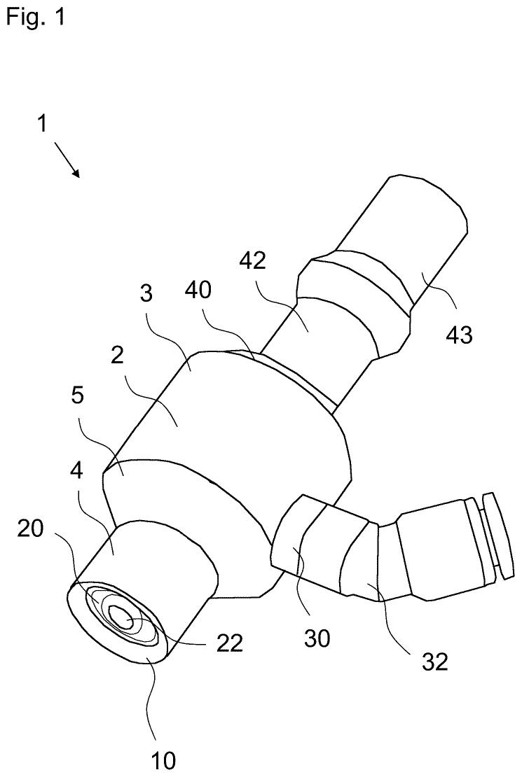

FIG. 1 illustrates a schematic simplified perspective drawing of an universal adapter for cleaning ports of a channel of a surgical instrument,

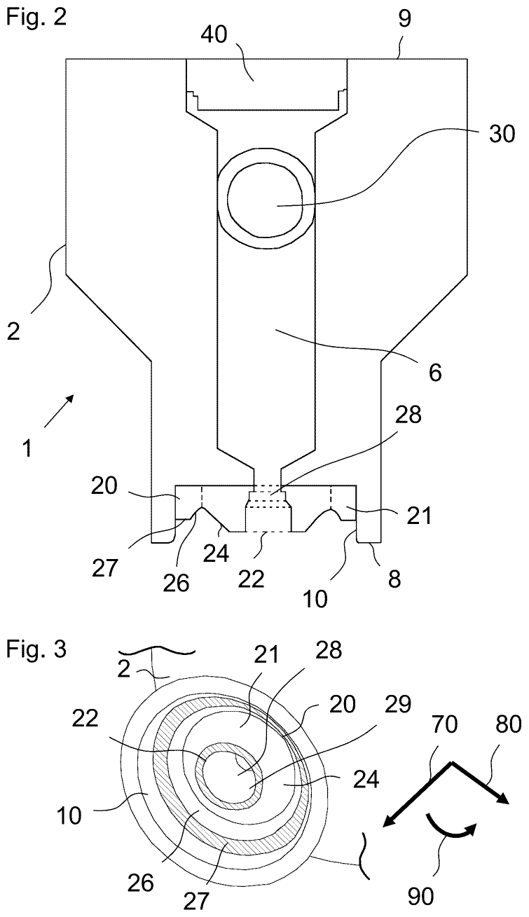

FIG. 2 illustrates a schematic simplified cross section of the universal adapter,

FIG. 3 illustrates a schematic simplified enlarged view of a port interface of the adapter,

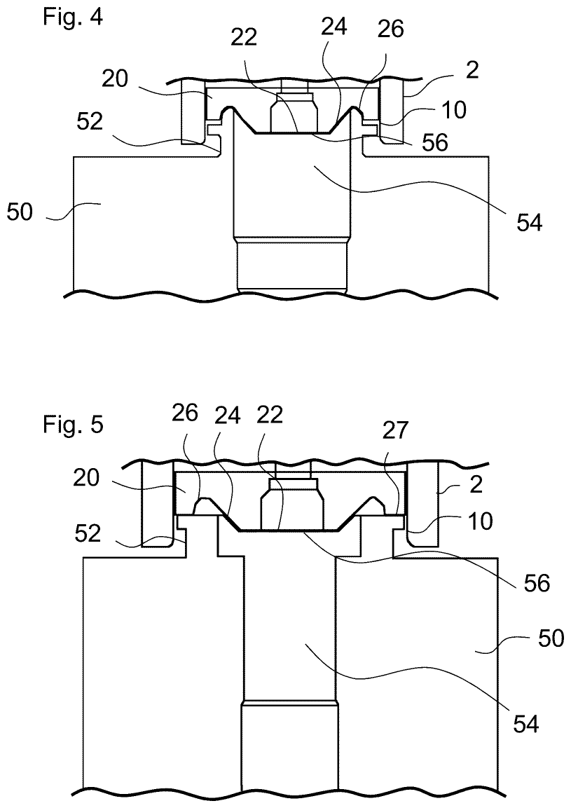

FIG. 4 illustrates a schematic simplified drawing of the adapter forming a seal with a first type of port,

FIG. 5 illustrates a schematic simplified drawing of the adapter forming a seal with a second type of port,

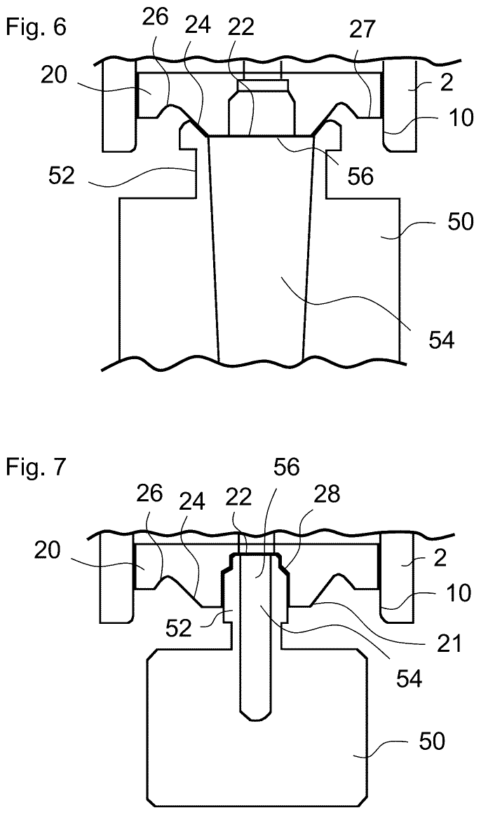

FIG. 6 illustrates a schematic simplified drawing of the adapter forming a seal with a third type of port and

FIG. 7 illustrates a schematic simplified drawing of the adapter forming a seal with a fourth type of port,

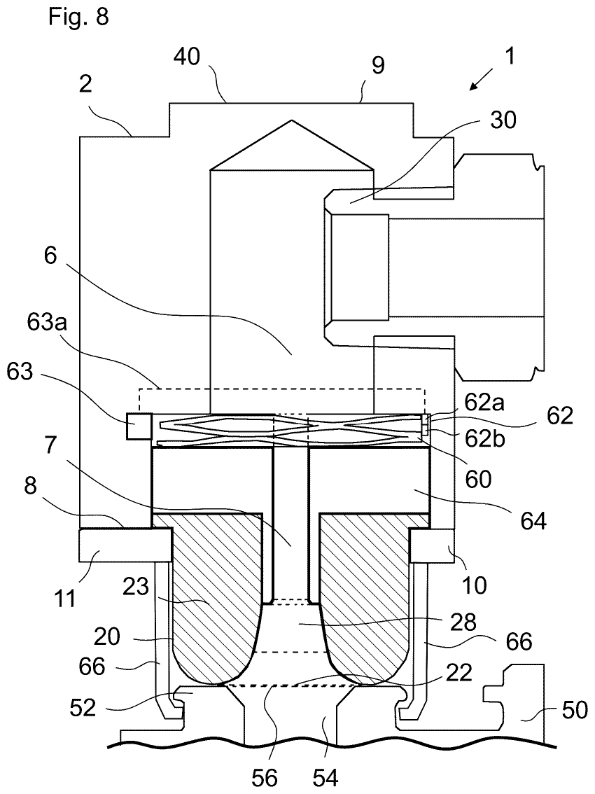

FIG. 8 illustrates an adapter for connecting a port of a channel of a surgical instrument to a cleaning fluid supply with an elastic seal with toroidal shape.

In the drawings, the same or similar types of elements or respectively corresponding parts are provided with the same reference numbers in order to prevent the item from needing to be reintroduced.

### DETAILED DESCRIPTION

In order to simplify the process of rinsing the channels of a surgical instrument with a cleaning fluid, the present embodiments provide an universal adapter to fit all types of ports of the channels of the surgical instrument or different types of surgical instruments. An example of such an universal adapter is shown in FIG. 1.

FIG. 1 shows a perspective drawing of an adapter 1 with a cylindrical housing 2. The housing 2 comprises a first cylinder 3, a smaller second cylinder 4 and a conical frustum 5 connecting the two cylinders 3, 4. A first base area 8 of the housing 2 is formed as a port interface 10 to connect different ports of surgical instruments 50. A gasket 20 with a central opening 22 is arranged inside the port interface 10. In order to supply the port interface 10 with cleaning fluid, a cleaning fluid connection 32 is connected to the adapter 1 via a connection interface 30, which is arranged on a lateral surface of the housing 2. The cleaning fluid may come from a cleaning fluid supply not shown in FIG. 1, for example a tank, and may consist of water, a mixture of water and a cleaning agent and/or a mixture of water and air. On a second base area 9 arranged on top of the first cylinder 3, there is arranged a pressure receiving area 40. This pressure receiving area 40 is configured to allow a user to exert a pressure or force on the adapter 1 to press it against a port 52 of a surgical instrument 50. In FIG. 1, a robot arm interface 42 is connected to the pressure receiving area 40, which allows a robot arm not shown in FIG. 1 to exert the necessary pressure or force on the adapter 1. The robot arm interface 42 may have the shape of a shaft 43. The robot arm may be attached to, or integral with, the shaft 43 or grab the shaft 43 to exert the necessary pressure or force on the adapter 1. The pressure receiving area 40 may comprise one or more holding devices not shown in FIG. 1, wherein the holding devices are configured to allow the user and/or the robot arm to grip the adapter 1.

FIG. 2 shows a cross sectional view of adapter 1. This view gives a better understanding of the gasket 20 arranged in the port interface 10 on the first base area 8 and the pressure receiving area 40 arranged on the second base area 9. The gasket 20 in this case is formed as a sealing lip 21. The sealing lip 21 comprises a ring shaped trim 27 surrounding an outer sealing surface 26, which in turn surrounds an inner sealing surface 24. The inner sealing surface 24 in turn surrounds the central opening 22, as also shown in the perspective drawing in FIG. 3. This figure illustrates how the different parts 27, 26, 24, 22 surround each other in the circumferential direction 90.

In order to better adapt to different ports, the outer sealing surface 26 is conical frustrum shaped and the inner sealing surface 24 is conical frustum shaped, such that a transition between the outer sealing surface 26 and inner sealing surface forms a concavity. The sealing lip 21 is, for example, made of silicone, which makes the gasket 20 elastic and deformable in the pressing direction 70 and the radial direction 80. This is also true for the gasket channel 28 inside the central opening 22, which is surrounded by a wall 29 made from the material of the sealing lip 21. An inner channel 6 connects the connection interface 30 with the gasket channel 28 and supplies cleaning fluid to the central opening 22. The gasket channel 28 widens towards the central opening 22, while the inner channel 6 gets narrower towards the gasket 20.

FIGS. 4, 5, 6 and 7 show the adapter 1 of FIGS. 2 and 3 connected to different ports 52 of one or more surgical instruments 50. In FIG. 4, the port interface 10 is connected to a channel 54 for air/water of a surgical instrument 50 in form of an endoscope. In order to fit the port 52 with its port opening 56, the gasket 20 is pressed on the edge of the port 52 at a position between the inner sealing surface 24 and the outer sealing surface 26. This allows the port interface 10 to form a sealing with the port 52, even though the port opening 56 is comparatively wide. The sealing formed this way may not be 100% waterproof. Instead about 10% of the cleaning fluid may escape from the sealing and rinse the outer parts of the port 52 and the surgical instrument 50. This is by design and helps with the cleaning of the outer surface. The other sealings shown in FIGS. 5, 6, and 7 may also not be 100% waterproof for the same reason.

FIG. 5 shows the adapter 1 connected to a port 52 belonging to a suction channel 54 of a surgical instrument 50 in form of an endoscope. The port opening 56 of the suction channel 54 is not quite as large as the port opening 56 of the air/water channel 54 in FIG. 4. Thus, only the inner sealing surface 24 extends into the channel 54, while the ring shaped trim 27 seals the port interface 10 to the edge of the port 52.

FIG. 6 shows the adapter 1 connected to an even smaller channel 54, which is a biopsy channel. For the port opening 56 of this channel 54, the inner sealing surface 24 fits into the port 52 in order to form the sealing. The outer sealing surface 26 and the ring shaped trim 27 do not contribute to the sealing for channels 54 of this size.

An even smaller channel 54 is shown in FIG. 7, which shows a surgical instrument 50 in form of a cystoscope or an accessory for a cystoscope. The port 52 of this channel 54 is so small, that it fits inside the central opening 22 and the gasket channel 28. Due to the elastic and deformable nature of the sealing lip 21, it can accommodate these types of ports 52 and form a sufficient sealing.

FIG. 8 shows a different type of universal adapter 1. The adapter 1 comprises a housing 2 of essentially cylindrical shape with a first base area 8 at its bottom and a second base area 9 at its top, where the pressure receiving area 40 is arranged. The connection interface 30 is again arranged on a lateral surface of the housing 2 and connected to the inner channel 6. The main difference is the form of the gasket 20, which is formed as an elastic seal 23 of toroidal shape. The elastic seal 23 is voluminous and made of a soft, compressible material, for example plastic or rubber.

The port interface 10 comprises an end plate 11, which fixes the elastic seal 23 to the housing 2 by overlapping edges of the elastic seal 23. The adapter 1 may also comprise a fixing device 66 to fix the adapter 1 to the port 52 of a surgical instrument 50, for example by screwing, snapping or clamping. The fixing device 66 can be a type of latching mechanism comprising multiple contact surfaces, which may be circular shaped. The fixing device 66 can also be a claw shaped device, as shown in FIG. 8. In both cases, the fixing device 66 keeps the port interface 10 connected to the port 52 in such a way that the channel 54 can be flushed with liquid from the adapter 1 without having to permanently keep it in position. The fixing device 66 may also be configured such that it releases the connection to the port 52 by pressing the adapter 1 slightly in the direction of the channel 54. When pressed on the port 52 in the pressing direction 70, the elastic seal 23 is compressed and forms a sealing. In order to prevent the gasket channel 28 from being narrowed or completely closed, a rigid tube 7 is arranged inside the central opening 22 of the elastic seal 23. The rigid tube 7 is encompassed by a guiding sleeve 64. Above the guiding sleeve 64 a spring 60 connects the elastic seal 23 to the housing 2 and absorbs excessive force acting on the elastic seal 23 from damaging the housing 2. A notification device 62 connected to the spring 60 may produce an acoustic signal when a force or pressure is greater than a predetermined threshold. The notification device 62 may comprise a sensor 62a for determining the force or pressure that outputs the acoustic signal to a processor 62b, as part of the notification device 62 or separately provided, which in turn controls a speaker 63 or a similar device to provide an audible warning. The speaker 63 is connected to the notification device 62 with a signal line 63a, which runs for example around the inner channel 6. The threshold is chosen to represent a sufficient sealing. The spring 60 may, for example, be a flat spring.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

1 adapter
2 housing
3 first cylinder
4 second cylinder
5 conical frustum
6 inner channel
7 rigid tube
8 first base area
9 second base area
10 port interface
11 end plate
20 gasket
21 sealing lip
22 central opening
23 elastic seal
24 inner sealing surface
26 outer sealing surface
27 ring shaped trim
28 gasket channel
29 wall
30 connection interface
32 cleaning fluid connection
40 pressure receiving area
42 robot arm interface
43 shaft
50 surgical instrument
52 port
54 channel
56 port opening
60 spring
62 notification device
62a sensor
62b processor
63 speaker
63a signal line
64 guiding sleeve
66 fixing device
70 pressing direction
80 radial direction
90 circumferential direction

What is claimed is:

1. An adapter for connecting a port of a channel of a surgical instrument to a cleaning fluid supply, the adapter comprising:
a rigid housing with a port interface and a connection interface for the cleaning fluid supply,
wherein the port interface comprises a gasket with a central opening for guiding the fluid into the port of the channel,
the port interface with the gasket is configured to be pressed along a pressing direction on the port of the channel to establish a sealing and rinse the channel with the cleaning fluid, and
the gasket is deformable along the pressing direction in order to fit differently configured ports.

2. The adapter according to claim 1, wherein the gasket comprises a flexible sealing lip, wherein the flexible sealing lip is configured to curve itself around the port opening of the port.

3. The adapter according to claim 2, wherein the flexible sealing lip comprises an outer sealing surface and an inner sealing surface, the outer sealing surface surrounding the inner sealing surface and the inner sealing surface surrounding the central opening in a circumferential direction.

4. The adapter according to claim 3, wherein the outer sealing surface is curved in an opposite direction as the inner sealing surface.

5. The adapter according to claim 3, wherein each of the outer sealing surface and the inner sealing surface have a conical frustum shape defining a concavity therebetween.

6. The adapter according to claim 3, wherein the outer sealing surface is surrounded by a ring shaped trim, and one or more of the inner sealing surface protrudes in the pressing direction from the trim and the outer sealing surface is recessed in the pressing direction compared to the trim.

7. The adapter according to claim 2, wherein the central opening is formed as a gasket channel extending in the pressing direction, and a wall of the gasket channel is flexible in a radial direction of the gasket.

8. The adapter according to claim 1, wherein the gasket comprises an elastic seal with a toroidal shape.

9. The adapter according to claim 8, wherein a height of a cross section of the elastic seal in the pressing direction is larger than a width of the cross section in a direction perpendicular to the pressing direction.

10. The adapter according to claim 8, wherein the elastic seal is compressible in the pressing direction to less than 80% of its length.

11. The adapter according to claim 8, wherein the elastic seal is compressible in the pressing direction to less than 50% of its length.

12. The adapter according to claim 1, further comprising a rigid tube extending through the central opening of the gasket.

13. The adapter according to claim 1, further comprising a spring connecting the housing to the gasket, wherein the spring is configured to absorb a force resulting from pressing the gasket on the port of the channel.

14. The adapter according to claim 13, wherein the spring is a flat spring.

15. The adapter according to claim 1, further comprising a notification device configured to output an acoustic signal when the gasket is pressed on the port of the channel with one or more of a force and pressure exceeding a predetermined threshold.

16. The adapter according to claim 1, wherein the gasket comprises one or more of silicone, plastic and rubber.

17. The adapter according to claim 1, wherein the gasket is configured to seal between the port interface and the port that is partially water-tight, wherein a water tightness of the seal is between 80% and 100%.

18. The adapter according to claim 17, wherein the water tightness of the seal is between 80% and 90%.

19. The adapter according to claim 1, wherein the housing has a cylindrical shape.

20. The adapter according to claim 19, wherein the cylindrical shape comprises a first cylinder connected to a second cylinder by a conical frustum, wherein a diameter of the first cylinder is larger than a diameter of the second cylinder.

21. The adapter according to claim 1, wherein the port interface is arranged in a first base area of the housing.

22. The adapter according to claim 1, wherein the connection interface for the cleaning fluid supply is arranged on a lateral surface of the housing.

23. The adapter according to claim 1, wherein the housing comprises a pressure receiving area at a second base area of the housing opposite the port interface, the pressure receiving area being configured to receive a pressing force to press the gasket on the port of the channel.

24. The adapter according to claim 23, wherein the pressure receiving area is configured as a robot arm interface.

25. The adapter according to claim 1, further comprising an inner channel extending inside the housing, the inner channel connecting the connection interface for the cleaning fluid supply with the port interface.

26. The adapter according to claim 25, wherein a diameter of the inner channel decreases in a direction towards the port interface.

27. The adapter according to claim 1, further comprising a fixing device, wherein the fixing device is configured to detachably fix the port interface to the port of the channel.
